(19) United States
(12) Patent Application Publication
(10) Pub. No.: US 2023/0360743 A1
(43) Pub. Date: Nov. 9, 2023

(54) SYSTEMS AND METHODS FOR IDENTIFYING LEAD CHEMICAL COMPOUNDS BASED ON REPRODUCED ORDER-DEPENDENT REPRESENTATIONS OF A CHEMICAL COMPOUND

(71) Applicant: Collaborative Drug Discovery, Inc., Burlingame, CA (US)

(72) Inventors: Peter Gedeck, Arlington, VA (US); Barry A. Bunin, Belmont, CA (US); William Michael Bowles, Redwood City, CA (US); Philip Cheung, San Diego, CA (US); Alex Michael Clark, Ottawa (CA)

(21) Appl. No.: 18/312,620

(22) Filed: May 5, 2023

Related U.S. Application Data
(60) Provisional application No. 63/338,487, filed on May 5, 2022.

(51) Int. Cl. G16C 20/70; G16C 20/30; G16C 20/20

(52) U.S. Cl. CPC G16C 20/70; G16C 20/30; G16C 20/20

(57) ABSTRACT

A method includes converting, by a generative network, an input into a latent vector representation of a sample chemical compound, wherein the input is one of an order-dependent representation of the sample chemical compound and a molecular graph representation of the sample chemical compound; determining, by an output neural network, one or more properties of the sample chemical compound based on the latent vector representation of the sample chemical compound; performing, by the output neural network, an optimization routine to select a candidate latent vector representation from among a plurality of latent vector representations based on the latent vector representation of the sample chemical compound, wherein the plurality of latent vector representations includes the latent vector representation of the sample chemical compound; and identifying, by the output neural network, a candidate chemical compound based on the candidate latent vector representation.

### CROSS-REFERENCE TO RELATED APPLICATIONS

This application claims priority to and the benefit of U.S. Provisional Patent Application No. 63/338,487, filed on May 5, 2022. The disclosure of the above application is incorporated herein by reference.

### GOVERNMENT LICENSE RIGHTS

This invention was made with government support under TR002527 awarded by the National Institutes of Health. The government has certain rights in the invention. 37 CFR 401.14(f)(4).

### FIELD

The present disclosure relates to systems and methods for identifying lead chemical compounds based on reproduced order-dependent representations of a chemical compound.

### BACKGROUND

The statements in this section merely provide background information related to the present disclosure and may not constitute prior art.

Chemical compounds may be represented using various notations and nomenclatures, such as an order-dependent representation (e.g., a simplified molecular-input line-entry system (SMILES) string), an order-independent representation (e.g., a Morgan Fingerprint), or a molecular graph representation. In some embodiments, autoencoder/decoder networks may be implemented to encode/convert the order-dependent representations into a numerical representation (e.g., a latent vector) and subsequently decode the numerical representation back into the order-dependent representations. However, multiple latent vectors may be generated for a given order-dependent representation, thereby making it difficult to train a predictive model that utilizes latent vectors to predict one or more properties of a given chemical compound.

### SUMMARY

This section provides a general summary of the disclosure and is not a comprehensive disclosure of its full scope or all of its features.

The present disclosure provides a method including converting, by a generative network, an input into a latent vector representation of a sample chemical compound, wherein the input is one of an order-dependent representation of the sample chemical compound and a molecular graph representation of the sample chemical compound; determining, by an output neural network, one or more properties of the sample chemical compound based on the latent vector representation of the sample chemical compound; performing, by the output neural network, an optimization routine to select a candidate latent vector representation from among a plurality of latent vector representations based on the latent vector representation of the sample chemical compound, wherein the plurality of latent vector representations includes the latent vector representation of the sample chemical compound; and identifying, by the output neural network, a candidate chemical compound based on the candidate latent vector representation.

In one embodiment, the optimization routine is one of a gradient descent routine, an iterative expansion routine, and a genetic algorithm routine.

In one embodiment, the optimization routine is the gradient descent routine, and wherein performing the gradient descent routine to select the candidate latent vector representation further comprises setting the latent vector representation of the sample chemical compound as an initial value of the gradient descent routine; descending along a gradient model of the plurality of latent vector representations to determine a gradient value of a given latent vector representation from among a remaining set of the plurality of latent vector representations; determining whether the gradient value satisfies a convergence condition; and designating the given latent vector representation as the candidate latent vector representation in response to the gradient value satisfying the convergence condition.

In one embodiment, the optimization routine is the iterative expansion routine, and wherein performing the iterative expansion routine to select the candidate latent vector representation further comprises setting the latent vector representation of the sample chemical compound as an initial value of the iterative expansion routine; selecting a given latent vector representation from among the plurality of latent vector representations that is proximate to the latent vector representation of the sample chemical compound; determining a gradient value of the given latent vector representation; determining whether the gradient value satisfies a convergence condition; and designating the given latent vector representation as the candidate latent vector representation in response to the gradient value satisfying the convergence condition.

In one embodiment, the optimization routine is the genetic algorithm routine, and wherein performing the genetic algorithm routine to select the candidate latent vector representation further comprises determining a fitness score for each latent vector representation of at least one set of the plurality of latent vector representations; selecting a given latent vector representation from among each of the at least one set based on the fitness score; performing, for each selected given latent vector representation, a reproduction routine to generate an additional latent vector representation; determining an additional fitness score associated with the additional latent vector representation and designating the additional latent vector representation as the candidate latent vector representation in response to the additional fitness score satisfying a convergence condition.

In one embodiment, the generative network further comprises a graph convolutional neural network and an input neural network.

In one embodiment, converting the input into the latent vector representation of the sample chemical compound further comprises generating, by the graph convolutional neural network, a graph of the sample chemical compound based on the input; and encoding the graph to generate the latent vector representation of the sample chemical compound based on at least one of an adjacency matrix of the graph convolutional neural network, one or more characteristics of the graph, one or more activation functions of the graph convolutional neural network, one or more node aggregation functions, and one or more weights of the graph convolutional neural network.

In one embodiment, the method further includes identifying one or more fragments and one or more substructures of the input; generating one or more nodes based on the one or more substructures; and generating one or more edges based on the one or more fragments, wherein the graph is further based on the one or more nodes and the one or more edges.

In one embodiment, the latent vector representation of the sample chemical compound is an order independent representation.

The present disclosure provides another method including converting, by a generative network, an input into a latent vector representation of a sample chemical compound, wherein the input is one of an order-dependent representation of the sample chemical compound and a molecular graph representation of the sample chemical compound, and wherein the latent vector representation of the sample chemical compound is an order independent representation; determining, by an output neural network, one or more properties of the sample chemical compound based on the latent vector representation of the sample chemical compound; performing, by the output neural network, an optimization routine to select a candidate latent vector representation from among a plurality of latent vector representations based on the latent vector representation of the sample chemical compound, wherein the plurality of latent vector representations includes the latent vector representation of the sample chemical compound, and wherein the optimization routine is one of a gradient descent routine, an iterative expansion routine, and a genetic algorithm routine; and identifying, by the output neural network, a candidate chemical compound based on the candidate latent vector representation.

The present disclosure provides a system including a generative network configured to convert an input into a latent vector representation of a sample chemical compound, wherein the input is one of an order-dependent representation of the sample chemical compound and a molecular graph representation of the sample chemical compound, and the latent vector representation of the sample chemical compound is an order independent representation. The system includes an output neural network configured to determine one or more properties of the sample chemical compound based on the latent vector representation of the sample chemical compound, perform an optimization routine to select a candidate latent vector representation from among a plurality of latent vector representations based on the latent vector representation of the sample chemical compound, wherein the plurality of latent vector representations includes the latent vector representation of the sample chemical compound, and wherein the optimization routine is one of a gradient descent routine, an iterative expansion routine, and a genetic algorithm routine, and identify a candidate chemical compound based on the candidate latent vector representation.

### BRIEF DESCRIPTION OF THE DRAWINGS

In order that the disclosure may be well understood, there will now be described various forms thereof, given by way of example, reference being made to the accompanying drawings, in which:

FIG. 1A illustrates a functional block diagram of a chemical compound system in accordance with the teachings of the present disclosure;

FIG. 1B illustrates a functional block diagram of a trained chemical compound system in accordance with the teachings of the present disclosure;

FIG. 2 illustrates a molecular graph representation and an order-dependent representation of a chemical compound in accordance with the teachings of the present disclosure;

FIG. 3 illustrates a graph of a chemical compound in accordance with the teachings of the present disclosure;

FIG. 4 illustrates a graph convolutional neural network in accordance with the teachings of the present disclosure;

FIG. 5A illustrates an example neural network in accordance with the teachings of the present disclosure;

FIG. 5B illustrates another example neural network in accordance with the teachings of the present disclosure;

FIG. 5C illustrates an additional example neural network in accordance with the teachings of the present disclosure;

FIG. 6 is a flowchart of an example control routine in accordance with the teachings of the present disclosure;

FIG. 7 illustrates an example output neural network in accordance with the teachings of the present disclosure; and

FIG. 8 is a flowchart of an example control routine in accordance with the teachings of the present disclosure.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

As described herein, the present disclosure provides systems and methods for generating a unique input representing a chemical compound and predicting, using a machine learning model, one or more properties of the chemical compound based on the input. To generate the unique input, the chemical compound system is trained to convert the input into a graph representing the chemical compound, encode the graph using a graph convolutional neural network to generate a latent vector representation of the chemical compound, and decode the latent vector representation based on a plurality of hidden states of a recurrent neural network to generate a reproduced order-dependent representation of the chemical compound.

The chemical compound system may be trained based on a comparison between an input (e.g., a latent vector representation of a sample chemical compound) and the corresponding reproduced order-dependent representation. That is, the chemical compound system may iteratively adjust one or more weights of a neural network until an aggregate loss value, which quantifies the difference between the input and the reproduced order-dependent representation, is less than a threshold value. Alternatively, the chemical compound system may be trained based on a comparison between one or more properties of the input and one or more properties associated with the corresponding reproduced order-dependent representation. That is, the chemical compound system may iteratively adjust one or more weights of a neural network until an aggregate loss value, which quantifies the difference between the property differences, is less than a threshold value

When the chemical compound system is trained, the chemical compound system is configured to generate or identify new chemical compounds that are related to the input. More specifically, the chemical compound system may include an output neural network that performs various optimization routines, such as a gradient descent routine, an iterative expansion routine, or a genetic algorithm routine, to identify or generate related chemical compounds related to the input. As such, the output neural network may reduce the amount of time needed during drug discovery for a medicinal chemist to modify a chemical compound and identify/generate a new lead compound to achieve a desired level of potency and other chemical/pharmacological properties (e.g., absorption, distribution, metabolism, excretion, toxicity, among others). Moreover, the trained chemical compound system enables medicinal chemists to explore chemical spaces similar to a given chemical compound more effectively, reduces failure rates for chemical compounds that advance through the drug discovery process, and accelerates the drug discovery process.

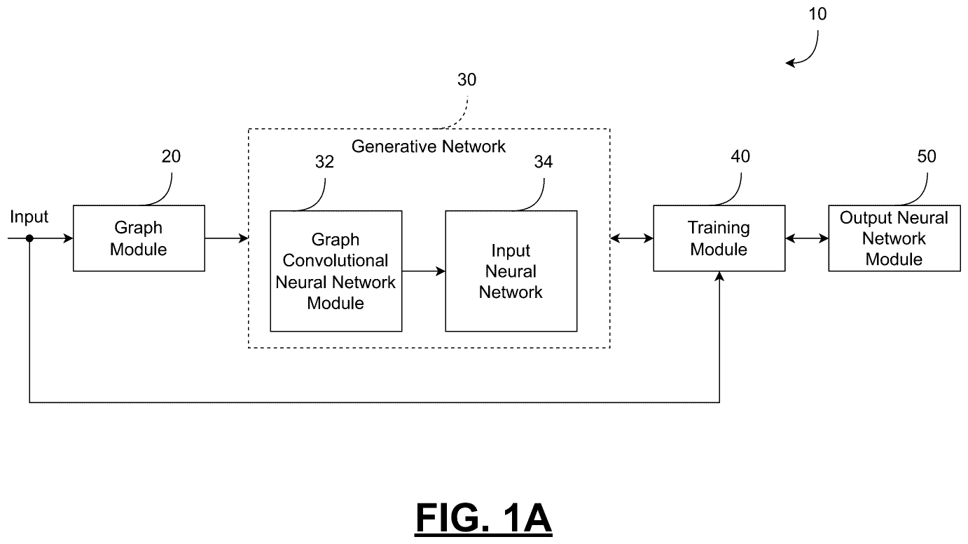

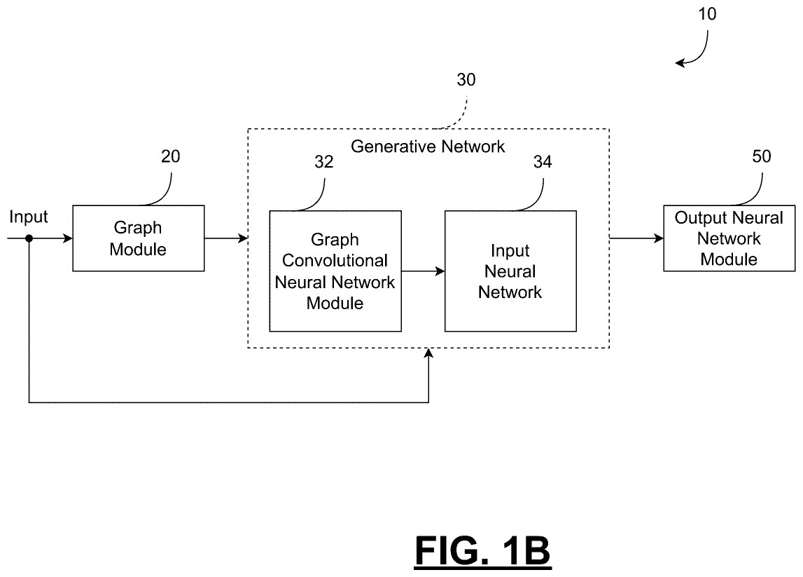

Referring to FIGS. 1A-1B, a functional block diagram of a chemical compound system 10 is shown and generally includes a graph module 20, a generative network 30, a training module 40, and an output neural network 50. While the components are illustrated as part of the chemical compound system 10, it should be understood that one or more components of the chemical compound system 10 may be positioned remotely from the chemical compound system 10. In one embodiment, the components of the chemical compound system 10 are communicably coupled using known wired/wireless communication protocols.

Referring to FIG. 1A, a functional block diagram of the chemical compound system 10 is shown operating during a training mode (i.e., the chemical compound system 10 includes the training module 40). In FIG. 1B, a functional block diagram of the chemical compound system 10 is shown during the chemical property prediction mode (i.e., the chemical compound system 10 is sufficiently trained and, as such, the training module 40 is removed from chemical compound system 10).

In one embodiment, the graph module 20 receives an input corresponding to at least one of an order-dependent representation of the chemical compound and a molecular graph representation of the chemical compound. As used herein, “order-dependent representation” refers to a nonunique text representation that defines the structure of the chemical compound. As an example, the order-dependent representation is a simplified molecular-input line-entry system (SMILES) string associated with the chemical compound, a DeepSMILES string, or a self-referencing embedded (SELFIE) string. As used herein, a “SMILES string” refers to a line notation that describes the corresponding structure using American Standard Code for Information Interchange (ASCII) strings. In one embodiment, the SMILES string may be one of a canonical SMILES string (i.e., the elements are the string are ordered in accordance with one or more canonical rules) and/or an isomeric SMILES string (i.e., the string defines isotopes, chirality, double bonds, and/or other properties of the chemical compound). It should be understood that the graph module 20 may receive other text-based representations of the chemical compound (e.g., a systematic name, a synonym, a trade name, a registry number, and/or an international chemical identifier (InChI)), and subsequently converted to an order-dependent representation based on, for example, a table that maps one or more-order dependent representations and the text-based representations.

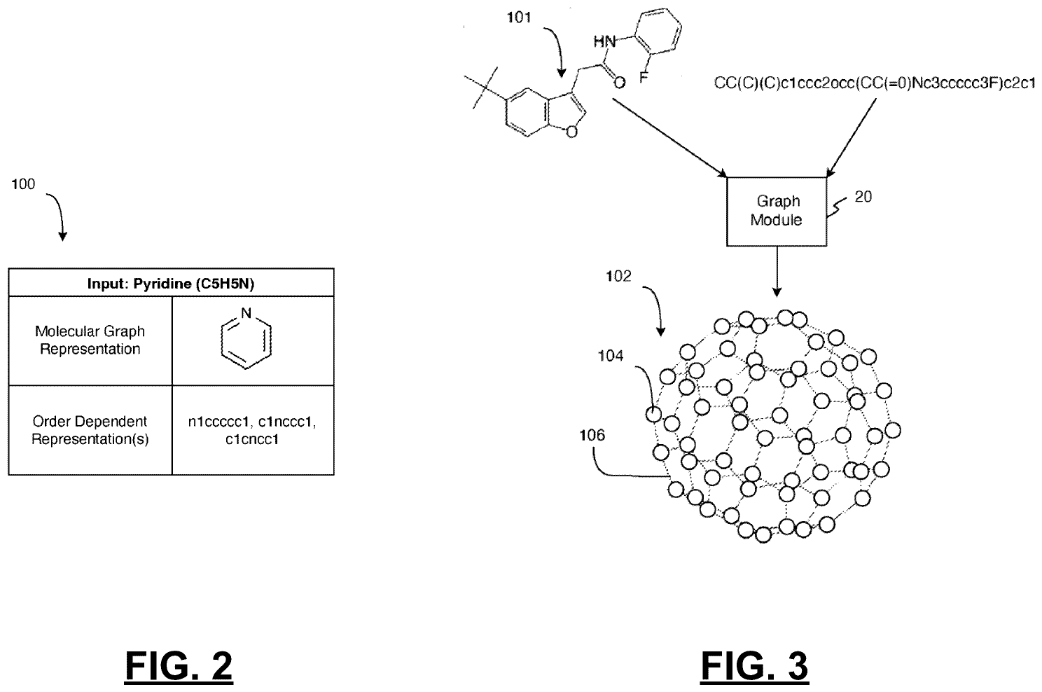

As used herein, the “molecular graph representation of the chemical compound” is a two-dimensional (2D) molecular graph that represents three-dimensional (3D) information of the chemical compound, such as atomic coordinates, bond angles, and chirality. In one embodiment, the 2D molecular graph is a tuple of a set of nodes and edges, where each edge connects pairs of nodes, and where each node is the set of all atoms of the chemical compound. As an example, and as shown in FIG. 2, the graph module 20 receives and/or generates an input 100 that is one of a molecular graph and/or order-dependent representation of pyridine. To perform the functionality described herein, the graph module 20 may include one or more interface elements (e.g., audio input and natural language processing systems, graphical user interfaces, keyboards, among other input systems) operable by the user to generate an input representing a given chemical compound.

In one embodiment and referring to FIGS. 1A-1B, the graph module 20 generates a graph of the chemical compound based on the input (i.e., at least one of the order-dependent representation and the molecular graph representation). As an example, the graph module 20 identifies one or more fragments and one or more substructures of the input. The one or more fragments of the input may include any fragment of the input, such as fragments connected to ring molecules of the input (e.g., monocycles or polycycles), fragments connected to amide bonds, fragments that identify a protein, fragments representing polymers or monomers, among others. The one or more substructures may include one or more combinations of fragments of the molecules, such as substituents and/or a moiety that collectively form a functional group.

Subsequently, the graph module 20 generates one or more nodes based on the substructures and one or more edges based on the one or more fragments, where the one or more nodes and one or more edges collectively form the graph. As a specific example and as shown in FIG. 3, the graph module 20 converts the SMILES string of 2-(5-tert-Butyl-1-benzofuran-3-yl)-N-(2-fluorophenyl)acetamide (e.g., CC(CXC)c1ccc2occ(CC(═O)Nc3ccccc3F)c2c1) or a corresponding molecular graph-based representation 101 to a graph 102 having a plurality of nodes 104 and edges 106. To perform the functionality described herein, the graph module 20 may perform known SMILES string to graph conversion routines that generate the graph 102 based on identified fragments and substructures of the SMILES string.

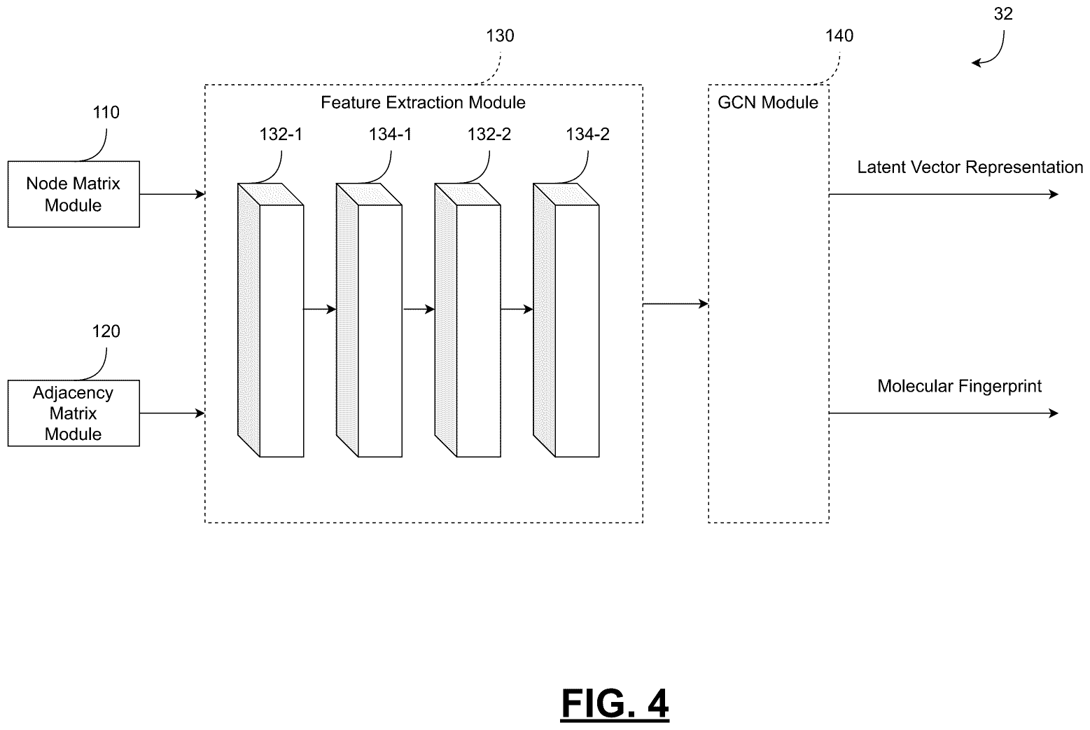

In one embodiment and referring to FIGS. 1 and 4, the generative network 30 includes a graph convolutional neural network (GCN) 32 and an input neural network 34. In one embodiment, the GCN 32 includes a node matrix module 110, an adjacency matrix module 120, a feature extraction module 130, and a GCN module 140. In one embodiment, the GCN 32 encodes the graph 102 based on at least one of a characteristic of the graph 102, an adjacency matrix defined by the adjacency matrix module 120, one or more node aggregation functions, an activation function performed by the feature extraction module 130, and one or more weights of the feature extraction module 130 to generate a latent vector representation of the chemical compound.

In one embodiment, the node matrix module 110 defines a node matrix based on the nodes 104 of the graph 102. As an example, the node matrix defines various atom features of the nodes 104, such as the atomic number, atom type, charge, chirality, ring features, hybridization, hydrogen bonding, aromaticity, among other atom features. To perform the functionality described herein, the node matrix module 110 may perform known input featurization routines to encode the atom features of the nodes 104 into the node matrix. In one embodiment, the adjacency matrix module 120 defines an adjacency matrix based on the edges 106 of the graph 102. In one embodiment, the adjacency matrix is a k×k matrix, where k is equal to the number of nodes 104, and where each element of the adjacency matrix indicates whether one of the edges 106 connects a given pair of nodes 104 of the graph 102.

In one embodiment, the feature extraction module 130 includes convolutional layers 132-1, 132-2 (collectively referred to hereinafter as “convolutional layers 132”) and activation layers 134-1, 134-2 (collectively referred to hereinafter as “activation layers 134”). While two convolutional layers 132 and two activation layers 134 are shown, it should be understood that the feature extraction module 130 may include any number of convolutional layers 132 and activation layers 134 in other forms and is not limited to the example described herein. It should also be understood that the feature extraction module 130 may also include other layers that are not shown, such as one or more pooling layers.

In one embodiment, the convolutional layers 132 are configured to perform a graph convolutional operation based on the node matrix and the adjacency matrix. As an example, at least one of the convolutional layers 132 performs one or more node aggregation functions, which comprise selecting an element from the node matrix corresponding to one of the nodes 104 and determining the atom features associated with the given node 104 and connected nodes (as defined by the adjacency matrix). The node aggregation function may also include performing a convolutional operation on the atom features associated with the given node 104 and the connected nodes to form a linear relationship between the given node 104 and the connected nodes and performing a pooling operation (e.g., a downsampling operation) to adjust the resolution of the linear relationship and generate one or more atom feature outputs. It should be understood that the node aggregation function may be performed for any number of elements of the node matrix (e.g., each element of the node matrix). As another example, at least one of the convolutional layers 132 performs an edge weight filtering routine that includes applying an edge feature matrix to at least one of the node matrix and the adjacency matrix, where the edge feature matrix defines one or more weights that selectively filter/adjust the atom feature values of the node matrix and/or adjacency matrix.

In one embodiment, the activation layers 134 are configured to perform an activation function on the one or more atom feature outputs of the convolutional layers 132 to learn one or more features of the nodes 104. Example activation functions include, but are not limited to, a sigmoid activation function, a tan-h activation function, a rectified linear unit function, among others.

In one embodiment, the GCN module 140 encodes the graph 102 into a latent vector representation by combining the one or more learned features associated with each of the nodes 104. As an example, the GCN module 140 performs known transformation operations to sum the one or more learned features associated with each of the nodes 104 and generate a fixed-size descriptor vector or a scale-invariant feature (SIFT) vector (as the latent vector representation). In one embodiment, the latent vector representation is an order-independent representation of the chemical compound. As used herein, “order-independent representation” refers to a uniquely defined textual or numerical representation of the structure of the chemical compound that is independent of any arbitrary ordering of the atoms. In one embodiment, the latent vector representation may also correspond to a given set of chemical and/or biological properties.

In one embodiment, the GCN module 140 generates a molecular fingerprint of the chemical compound based on the latent vector representation of the chemical compound and known latent vector to molecular fingerprint conversion routines. Example molecular fingerprints include, but are not limited to, a Morgan fingerprint, a hashed-based fingerprint, an atom-pair fingerprint, among other known molecular fingerprints. As described below in further detail, the training module 40 is configured to train the GCN 32 and/or the input neural network 34 based on the molecular fingerprint and/or the latent vector representation.

In one embodiment, the input neural network 34 is a recurrent neural network, but it should be understood that the input neural network 34 may employ a convolutional neural network in other forms. The input neural network 34 decodes the latent vector representation generated by the GCN 32 based on a plurality of hidden states of the recurrent neural network to generate a reproduced order-dependent representation of the chemical compound.

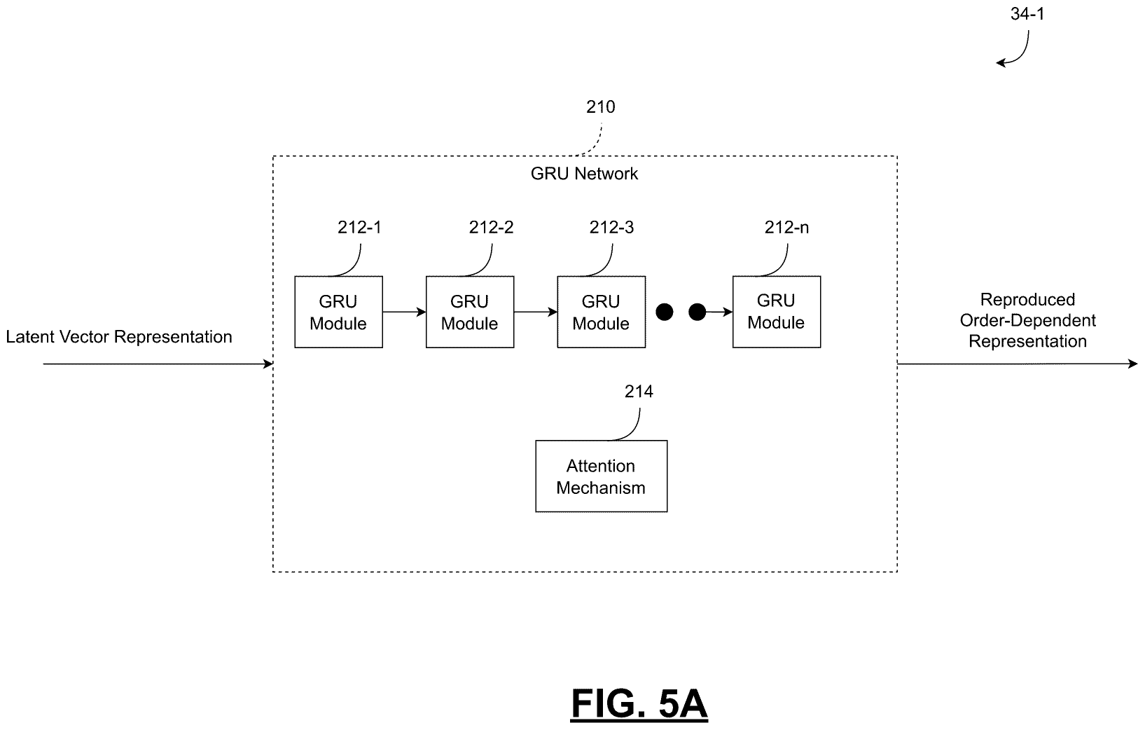

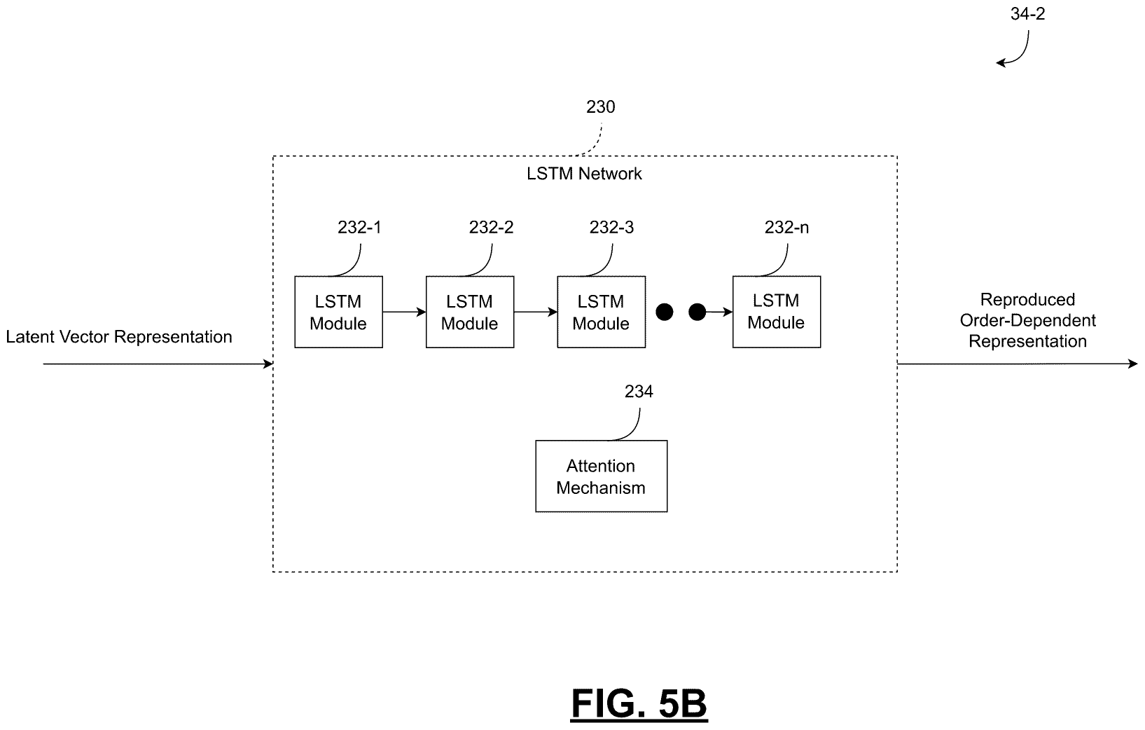

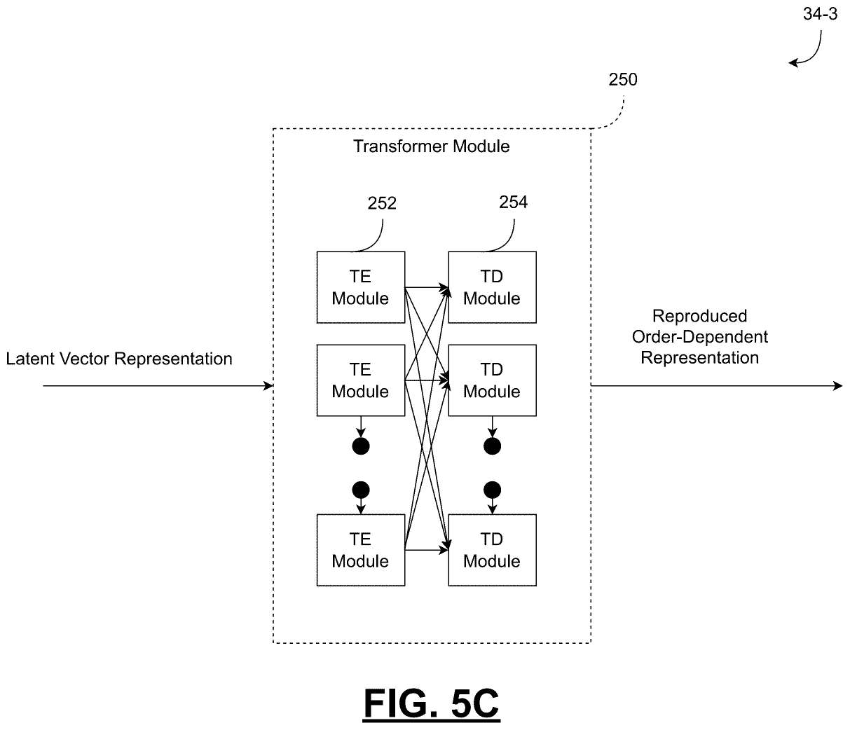

As an example, and as shown in FIG. 5A, input neural network 34-1 (as the input neural network 34) is a gated recurrent unit (GRU) network 210 and includes gated recurrent unit modules 212-1, 212-2, 212-3, . . . 212-n (collectively referred to hereinafter as “GRU modules 212”) and an attention mechanism 214. It should be understood that the GRU network 210 may include any number of GRU modules 212 in other forms and is not limited to the example described herein. It should also be understood that the attention mechanism 214 may be removed from the GRU network 210. Furthermore, it should be understood that the GRU modules 212 may be replaced with a plurality of ungated recurrent units (not shown) in other forms.

In one embodiment, each of the GRU modules 212 generates an output vector (hᵥ₊₁) based on an update gate vector (zᵥ), a reset gate vector (rᵥ), a hidden state vector (h′ᵥ), and the following relations:

zᵥ=σ(W_{z}xᵥ+U_{z}aᵥ+V_{z}cᵥ+b_{z}) (1)

rᵥ=σ(Wᵣxᵥ+Uᵣaᵥ+Vᵣcᵥ+bᵣ) (2)

h′ᵥ=tanh(W(rᵥ⊙hᵥ)+Uaᵥ+Vcᵥ+bₕ) (3)

hᵥ₊₁=(1−zᵥ)⊙hᵥzᵥ⊙h′ᵥ (4)

In relations (1)-(4), W_{z}, Wᵣ, U_{z}, and Uᵣ are input weights of the update gate vector and reset gate vectors, W is a weight of the GRU module 212, xᵥ is an input representing one or more elements of the latent vector, aᵥ is a hidden state value (i.e., the reset gate vector depends on the hidden state of the preceding GRU module 212), cᵥ is a conditioning value, b_{z}, bᵣ, bₕ are bias values, “V” is a matrix that is based on a predefined hidden dimension and the latent vector representation, and “a” is a sigmoid function. In one embodiment, the update gate vector indicates whether the GRU module 212 updates and/or preserves the hidden state value, and the reset gate vector indicates whether the GRU module 212 utilizes the previous hidden state value to calculate the hidden state vector and the output vector.

Specifically, the GRU modules 212 decode the latent vector representation based on the hidden states of the GRU modules 212 to generate a token-based representation of the chemical compound having one or more tokens. As used herein, “tokens” refer to one or more characters of the order-dependent representation, such as one or more characters of the SMILES string. In one embodiment, the GRU modules 212 decode the latent vector representation and generate the token-based representation of the chemical compound one token at a time.

As an example, the first GRU module 212-1 generates the first token based on the latent vector representation and a trainable starting state, and the first token may be a beginning-of-sequence (BOS) token that initiates the GRU modules 212. In some embodiments, the first GRU module 212-1 is further configured to encode the latent vector representation with latent vector conditioning routine based on an encoding routine (e.g., one-hot encoding routine) and an embedding routine, thereby enabling the first GRU module 212-1 to initialize the hidden state of the GRU modules 212. After producing the first token, the second GRU module 212-2 generates a second token based on the hidden state of the first GRU module 212-1 and the latent vector representation. After producing the second token, the third GRU module 212-3 generates a third token based on the hidden state of the second GRU module 212-2 and the latent vector representation. The GRU modules 212 collectively and recursively generate tokens until the last GRU module 212-n produces an end-of-sequence (EOS) token. In one embodiment, the GRU module 212-n aggregates each of the generated tokens to generate the reproduced order-dependent representation of the chemical compound.

In one embodiment, the attention mechanism 214 instructs each of the GRU modules 212 to generate the respective token based on each previous hidden states. As an example, and after producing the second token, the third GRU module 212-3 generates a third token based on the hidden state of the first and second GRU modules 212-1, 212-2 and the latent vector representation. As another example, the nth GRU module 212-n generates the EOS token based on the hidden state of each of the preceding GRU modules 212 and the latent vector representation.

As another example and as shown in FIG. 5B, input neural network 34-2 (as the input neural network 34) is a long short-term memory (LSTM) network 230 and includes LSTM modules 232-1, 232-2, 232-3 . . . 232-n (collectively referred to hereinafter as “LSTM modules 232”) and an attention mechanism 234. It should be understood that the LSTM network 230 may include any number of LSTM modules 232 in other forms and is not limited to the example described herein. In one embodiment, the LSTM modules 232 are configured to perform similar functions as the GRU modules 212, but in this form, LSTM modules 232 are configured to calculate input vectors, output vectors, and forget vectors based on the hidden states of the LSTMs and the latent vector representation to generate the reproduced order-dependent representation of the chemical compound. In one embodiment, the attention mechanism 234 is configured to perform similar operations as the attention mechanism 214 described above.

As an additional example and as shown in FIG. 5C, input neural network 34-3 (as the input neural network 34) is a transformer 250 and includes transformer encoder modules 252-1, 252-2, . . . 252-n (collectively referred to hereinafter as “TE modules 252”) and transformer decoder modules 254-1, 254-2, . . . 254-n (collectively referred to hereinafter as “TD modules 254”). In one embodiment, the TE modules 252 each include feed-forward and self-attention layers that are collectively configured to encode a portion of the latent vector representation. The TD modules 254 each include feed-forward, self-attention, and encoder-decoder attentional layers that collectively decode each of the encoded latent vector representation portions generated by the TE modules 252 to generate the reproduced order-dependent representation of the chemical compound.

In one embodiment, the training module 40 is configured to train a machine learning model (e.g., the generative network 30) based on at least one of the input, the reproduced order-dependent representation, the latent vector representation, and the molecular fingerprint. As an example, the training module 40 is configured to determine an aggregate loss value based on a loss function that derives the difference between, for example, the input and the reproduced order-dependent representation and/or the input and the molecular fingerprint. In some embodiments, the loss function includes a regularization variable that prevents memorization and overfitting problems associated with larger weights of the GCN 32 and/or the input neural network 34. Accordingly, the training module 40 may iteratively adjust one or more weights of the feature extraction module 130 of the GCN 32 and/or one or more weights of the input neural network 34 (e.g., the weights of the GRU modules 212) until the aggregate loss value is less than a threshold value.

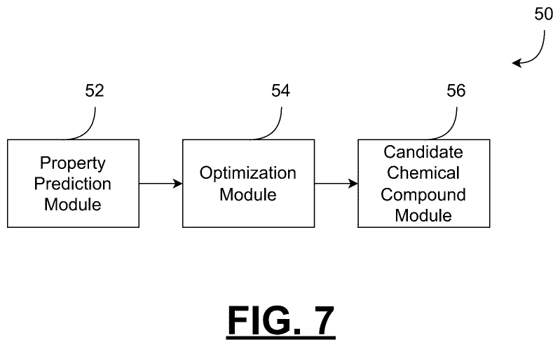

As another example, the training module 40 instructs the output neural network 50 to determine one or more statistical properties of the latent vector representation (described below in further detail with reference to FIG. 7). The training module 40 may determine an aggregate loss value based on a loss function that quantifies the difference between the determined statistical properties and known statistical properties associated with the input. Accordingly, the training module 40 may iteratively adjust one or more weights of the feature extraction module 130 of the GCN 32 and/or one or more weights of the input neural network 34 (e.g., the weights of the GRU modules 212) until the aggregate loss value associated with the statistical properties is less than a threshold value.

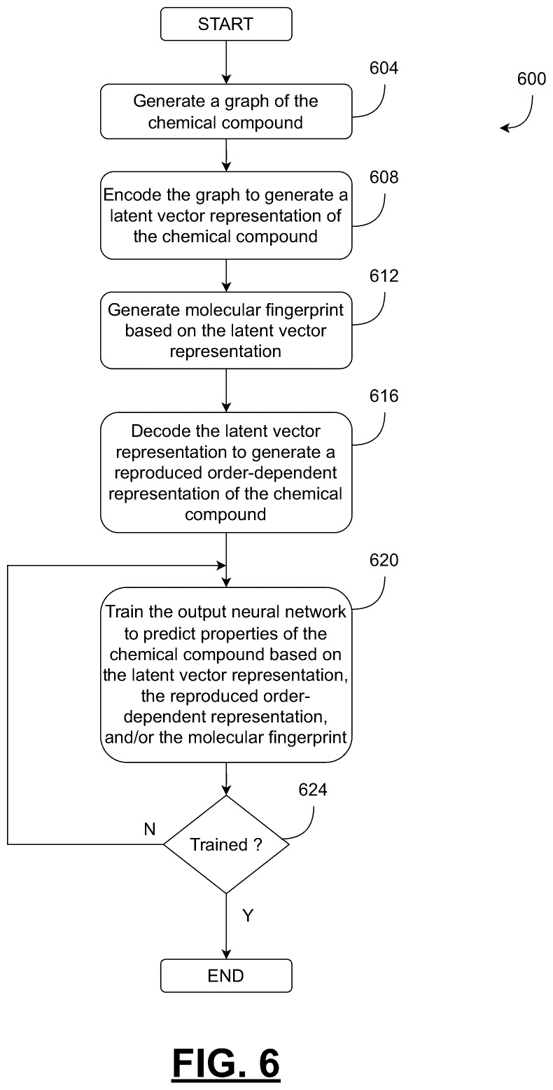

Referring to FIG. 6, a routine 600 for defining the generative network 30 is shown. At 604, the graph module 20 generates a graph of the chemical compound. At 608, the generative network 30 encodes the graph to generate a latent vector representation of the chemical compound. At 612, the generative network 30 generates a molecular fingerprint based on the latent vector representation. At 616, the generative network 30 decodes the latent vector representation to generate a reproduced order-dependent representation of the chemical compound. At 620, the training module 40 trains the output neural network 50 to predict properties of the chemical compound based on the latent vector representation, the reproduced order-dependent representation, and/or the molecular fingerprint. At 624, the training module 40 determines whether the output neural network 50 is trained based on the loss function. If the output neural network 50 is trained, the routine ends. Otherwise, the routine 600 proceeds to 620.

Referring back to FIG. 1, the generative network 30 is configured to, when trained (as described above with reference to FIG. 6), accurately convert an input corresponding to a sample chemical compound (e.g., the order-dependent representation or the molecular-graph representation) into a corresponding latent vector representation. Subsequently, the output neural network 50 is configured to predict various chemical properties of the input, generate/identify new chemical compounds that are related to the input, and/or filter chemical compounds that are unrelated to the input and/or have a statistical property that deviate from the input beyond a threshold amount.

Specifically, and referring to FIG. 7, the output neural network 50 includes a property prediction module 52, an optimization module 54, and a candidate chemical compound module 56. In one embodiment, the property prediction module 52 is configured to determine one or more properties of the sample chemical compound based on the latent vector representation of the sample chemical compound (also referred to as “sample latent vector representation”) obtained from the generative network 30. As an example, the property prediction module 52 employs a known multilayer perceptron networks or a regression model to predict the properties of the sample chemical compound based on the latent vector representation. Example properties include, but are not limited to, a water-octanal partition coefficient (log P), a synthetic accessibility score (SAS), a qualitative estimate of drug-likeness (QED), a natural-product (NP) score, absorption, distribution, metabolism, excretion, toxicity, among other properties of the latent vector representation of the sample chemical compound.

In one embodiment, the optimization module 54 is configured to perform an optimization routine to select, based on the sample latent vector representation, a candidate latent vector representation from among a plurality of latent vector representations. That is, the optimization module 54 is configured to explore the latent chemical space that is similar to the sample chemical compound to thereby generate or identify new and related chemical compounds. Example optimization routines include, but are not limited to, a gradient descent routine, an iterative expansion routine, and a genetic algorithm routine.

As an example, the gradient descent routine may include setting the sample chemical compound latent vector representation and a corresponding property to an initial value of a gradient model of the gradient descent routine. In one embodiment, the gradient model includes a plurality of data points that correspond to a plurality of latent vector representations having a given property that deviates from the property of the sample chemical compound latent vector representation within a given threshold. As an example, the gradient model includes a plurality of latent vector representations having a water-octanal partition coefficient that deviates from the initial value by a predetermined log value.

In response to setting the sample chemical compound latent vector representation as the initial value, the optimization module 54 descends along the gradient model in accordance with a given step size to determine a gradient value of another latent vector representation of the gradient model. If the gradient value satisfies a convergence condition, the optimization module 54 designates the given latent vector representation as a candidate latent vector representation. Otherwise, the optimization module 54 iteratively descends the gradient model to identify a latent vector representation that satisfies the convergence condition. In one embodiment, the convergence condition is satisfied when, for example, step size changes along the gradient descent model result in a value change of the given property that is less than a given threshold value change. In one embodiment, the optimization module 54 may employ known gradient descent convergence calculation routines to determine whether the convergence condition is satisfied.

As another example and like the gradient descent routine, the iterative expansion routine may include setting the sample chemical compound latent vector representation and a corresponding property to an initial value of the gradient model. In response to setting the latent vector representation as the initial value, the optimization module 54 arbitrarily or randomly selects a set of latent vector representation of the gradient model that is proximate to (i.e., adjacent and/or near) the initial value. If the largest gradient value of the selected set satisfies the convergence condition (as described above), the optimization module 54 designates the given latent vector representation as the candidate latent vector representation. Otherwise, the optimization module 54 iteratively selects a new set of latent vector representations that are proximate to one of the currently selected latent vector representations of the gradient model until the convergence condition is satisfied.

As an additional example, the genetic algorithm routine may include setting the sample chemical compound latent vector representation and a corresponding property to an initial value of a genetic algorithm model. In one embodiment, the genetic algorithm model includes a plurality of data points that correspond to a plurality of latent vector representations having a given property that deviates from the property of the sample chemical compound latent vector representation within a given threshold. As an example, the genetic algorithm model includes a plurality of latent vector representations having a toxicity value that deviates from the initial value by a predetermined amount.

In response to setting the sample chemical compound latent vector representation as the initial value, the optimization module 54 randomly or arbitrarily selects a set of latent vector representations from the genetic algorithm model and determines a fitness score associated with each of the selected latent vector representations. In one embodiment, the fitness score correlates to a degree of matching to a desired property value (e.g., a desired toxicity). Subsequently, the optimization module 54 further selects a subset of latent vector representations from among the set having the highest fitness scores and performs a reproduction routine (e.g., a crossover routine or a mutation routine) to generate an additional latent vector representation based on the subset of latent vector representations.

Furthermore, the optimization module 54 determines an additional fitness score for the additional latent vector representation and determines whether the additional fitness score satisfies the convergence condition. If the convergence condition is satisfied, the optimization module 54 designates the additional latent vector representation as the candidate latent vector representation. Otherwise, the optimization module 54 iteratively repeats the genetic algorithm based on the current additional latent vector representation until the convergence condition is satisfied. In one embodiment, the convergence condition is satisfied when, for example, step size changes among consecutively generated additional latent vector representations result in a value change of the given property that is less than a given threshold value change, and the optimization module 54 may employ known genetic algorithm descent convergence calculation routines to determine whether the convergence condition is satisfied. In another embodiment, the convergence condition of the genetic algorithm routine is satisfied when a predetermined number of iterations of the genetic algorithm routine is performed.

As such, the optimization routines described herein may identify a latent vector representation that is associated with a candidate chemical compound that may be suitable as a lead chemical compound for further exploration and testing when developing new drugs. Specifically, the candidate chemical compound module 56 may perform known decoding routines to convert the latent vector representation of the identified candidate chemical compound into a molecular graph or text representation of the candidate chemical compound, thereby enabling a medicinal chemist to identify the corresponding candidate chemical compound. In some embodiments, the candidate chemical compound module 56 may perform known retrosynthetic analysis routines to determine whether the fabrication of the candidate chemical compound is feasible. Accordingly, the optimization routines may be iteratively performed until the feasibility value is determined to be sufficient or satisfies other qualitative or quantitative conditions.

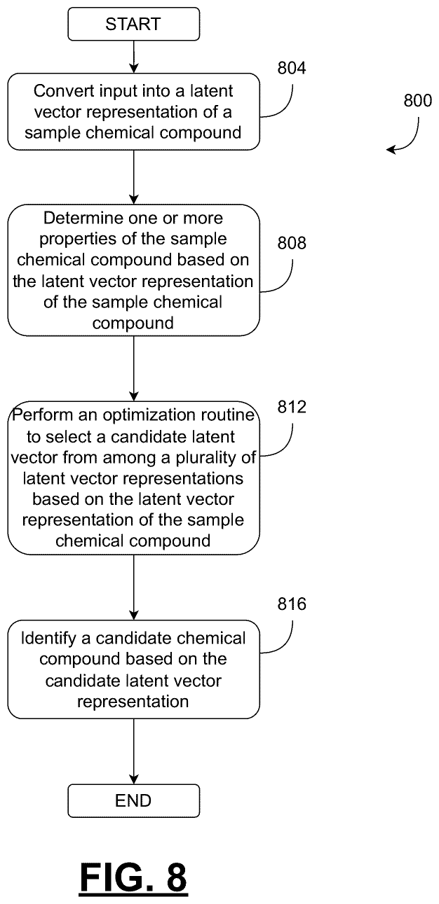

Referring to FIG. 8, a routine 800 is shown for exploring a chemical latent space. By performing the routine 800, medicinal chemists can explore the chemical space similar to a sample chemical compound and select a lead candidate series more effectively, the failure rates for chemical compounds that advance through the drug discovery process are reduced, and the drug discovery process is accelerated. At 804, the generative network 30 converts an input into a latent vector representation of a sample chemical compound. At 808, the output neural network 50 determines one or more properties of the sample chemical compound based on the latent vector representation of the sample chemical compound. At 812, the output neural network performs an optimization routine to select a candidate latent vector representation from among a plurality of latent vector representations based on the latent vector representation of the sample chemical compound. At 816, the output neural network 50 identifies a candidate chemical compound based on the candidate latent vector representation.

The generative network 30 and the output neural network 50 described herein may be configured to transform a memory of a computer system to include one or more data structures, such as, but not limited to, arrays, extensible arrays, linked lists, binary trees, balanced trees, heaps, stacks, and/or queues. These data structures can be configured or modified through the rule generation/adjudication process and/or the training process to improve the efficiency of a computer system when the computer system operates in an inference mode to make an inference, prediction, classification, suggestion, or the like with respect to generating reproduced order-dependent representations and selecting candidate latent vector representations based on an input.

The description of the disclosure is merely exemplary in nature. Thus, variations that do not depart from the substance of the disclosure are intended to be within the scope of the disclosure. Such variations are not to be regarded as a departure from the spirit and scope of the disclosure.

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information, but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, the term module may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality, such as, but not limited to, transceivers, routers, input/output interface hardware, among others; or a combination of some or all of the above, such as in a system-on-chip.

The term memory is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The term code, as used below, may include software, firmware, and/or microcode, and may refer to computer programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As an example, source code may be written using syntax from languages including C, C++, C#, Objective-C, Swift, Haskell, Go, SQL, R, Lisp, Java®, Fortran, Perl, Pascal, Curd, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

That which is claimed is:

1. A method comprising:
converting, by a generative network, an input into a latent vector representation of a sample chemical compound, wherein the input is one of an order-dependent representation of the sample chemical compound and a molecular graph representation of the sample chemical compound;
determining, by an output neural network, one or more properties of the sample chemical compound based on the latent vector representation of the sample chemical compound;
performing, by the output neural network, an optimization routine to select a candidate latent vector representation from among a plurality of latent vector representations based on the latent vector representation of the sample chemical compound, wherein the plurality of latent vector representations includes the latent vector representation of the sample chemical compound; and
identifying, by the output neural network, a candidate chemical compound based on the candidate latent vector representation.

2. The method of claim 1, wherein the optimization routine is one of a gradient descent routine, an iterative expansion routine, and a genetic algorithm routine.

3. The method of claim 2, wherein the optimization routine is the gradient descent routine, and wherein performing the gradient descent routine to select the candidate latent vector representation further comprises:
setting the latent vector representation of the sample chemical compound as an initial value of the gradient descent routine;
descending along a gradient model of the plurality of latent vector representations to determine a gradient value of a given latent vector representation from among a remaining set of the plurality of latent vector representations;
determining whether the gradient value satisfies a convergence condition; and
designating the given latent vector representation as the candidate latent vector representation in response to the gradient value satisfying the convergence condition.

4. The method of claim 2, wherein the optimization routine is the iterative expansion routine, and wherein performing the iterative expansion routine to select the candidate latent vector representation further comprises:
setting the latent vector representation of the sample chemical compound as an initial value of the iterative expansion routine;
selecting a given latent vector representation from among the plurality of latent vector representations that is proximate to the latent vector representation of the sample chemical compound;
determining a gradient value of the given latent vector representation;
determining whether the gradient value satisfies a convergence condition; and
designating the given latent vector representation as the candidate latent vector representation in response to the gradient value satisfying the convergence condition.

5. The method of claim 2, wherein the optimization routine is the genetic algorithm routine, and wherein performing the genetic algorithm routine to select the candidate latent vector representation further comprises:
determining a fitness score for each latent vector representation of at least one set of the plurality of latent vector representations;
selecting a given latent vector representation from among each of the at least one set based on the fitness score;
performing, for each selected given latent vector representation, a reproduction routine to generate an additional latent vector representation;
determining an additional fitness score associated with the additional latent vector representation; and
designating the additional latent vector representation as the candidate latent vector representation in response to the additional fitness score satisfying a convergence condition.

6. The method of claim 1, wherein the generative network further comprises a graph convolutional neural network and an input neural network.

7. The method of claim 6, wherein converting the input into the latent vector representation of the sample chemical compound further comprises:
generating, by the graph convolutional neural network, a graph of the sample chemical compound based on the input; and
encoding the graph to generate the latent vector representation of the sample chemical compound based on at least one of an adjacency matrix of the graph convolutional neural network, one or more characteristics of the graph, one or more activation functions of the graph convolutional neural network, one or more node aggregation functions, and one or more weights of the graph convolutional neural network.

8. The method of claim 7 further comprising:
identifying one or more fragments and one or more substructures of the input;
generating one or more nodes based on the one or more substructures; and
generating one or more edges based on the one or more fragments, wherein the graph is further based on the one or more nodes and the one or more edges.

9. The method of claim 1, wherein the latent vector representation of the sample chemical compound is an order independent representation.

10. A method comprising:
converting, by a generative network, an input into a latent vector representation of a sample chemical compound, wherein the input is one of an order-dependent representation of the sample chemical compound and a molecular graph representation of the sample chemical compound, and wherein the latent vector representation of the sample chemical compound is an order independent representation;
determining, by an output neural network, one or more properties of the sample chemical compound based on the latent vector representation of the sample chemical compound;
performing, by the output neural network, an optimization routine to select a candidate latent vector representation from among a plurality of latent vector representations based on the latent vector representation of the sample chemical compound, wherein the plurality of latent vector representations includes the latent vector representation of the sample chemical compound, and wherein the optimization routine is one of a gradient descent routine, an iterative expansion routine, and a genetic algorithm routine; and
identifying, by the output neural network, a candidate chemical compound based on the candidate latent vector representation.

11. The method of claim 10, wherein the optimization routine is the gradient descent routine, and wherein performing the gradient descent routine to select the candidate latent vector representation further comprises:
setting the latent vector representation of the sample chemical compound as an initial value of the gradient descent routine;
descending along a gradient model of the plurality of latent vector representations to determine a gradient value of a given latent vector representation from among a remaining set of the plurality of latent vector representations;
determining whether the gradient value satisfies a convergence condition; and
designating the given latent vector representation as the candidate latent vector representation in response to the gradient value satisfying the convergence condition.

12. The method of claim 10, wherein the optimization routine is the iterative expansion routine, and wherein performing the iterative expansion routine to select the candidate latent vector representation further comprises:
setting the latent vector representation of the sample chemical compound as an initial value of the iterative expansion routine;
selecting a given latent vector representation from among the plurality of latent vector representations that is proximate to the latent vector representation of the sample chemical compound;
determining a gradient value of the given latent vector representation;
determining whether the gradient value satisfies a convergence condition; and
designating the given latent vector representation as the candidate latent vector representation in response to the gradient value satisfying the convergence condition.

13. The method of claim 10, wherein the optimization routine is the genetic algorithm routine, and wherein performing the genetic algorithm routine to select the candidate latent vector representation further comprises:
determining a fitness score for each latent vector representation of at least one set of the plurality of latent vector representations;
selecting a given latent vector representation from among each of the at least one set based on the fitness score;
performing, for each selected given latent vector representation, a reproduction routine to generate an additional latent vector representation;
determining an additional fitness score associated with the additional latent vector representation; and
designating the additional latent vector representation as the candidate latent vector representation in response to the additional fitness score satisfying a convergence condition.

14. The method of claim 10, wherein the generative network further comprises a graph convolutional neural network and an input neural network.

15. The method of claim 14, wherein converting the input into the latent vector representation of the sample chemical compound further comprises:
generating, by the graph convolutional neural network, a graph of the sample chemical compound based on the input; and
encoding the graph to generate the latent vector representation of the sample chemical compound based on at least one of an adjacency matrix of the graph convolutional neural network, one or more characteristics of the graph, one or more activation functions of the graph convolutional neural network, one or more node aggregation functions, and one or more weights of the graph convolutional neural network.

16. The method of claim 15 further comprising:
identifying one or more fragments and one or more substructures of the input;
generating one or more nodes based on the one or more substructures; and
generating one or more edges based on the one or more fragments, wherein the graph is further based on the one or more nodes and the one or more edges.

17. A system comprising:
a generative network configured to convert an input into a latent vector representation of a sample chemical compound, wherein:
the input is one of an order-dependent representation of the sample chemical compound and a molecular graph representation of the sample chemical compound, and
the latent vector representation of the sample chemical compound is an order independent representation; and
an output neural network configured to:
determine one or more properties of the sample chemical compound based on the latent vector representation of the sample chemical compound;
perform an optimization routine to select a candidate latent vector representation from among a plurality of latent vector representations based on the latent vector representation of the sample chemical compound, wherein the plurality of latent vector representations includes the latent vector representation of the sample chemical compound, and wherein the optimization routine is one of a gradient descent routine, an iterative expansion routine, and a genetic algorithm routine; and
identify a candidate chemical compound based on the candidate latent vector representation.

18. The system of claim 17, wherein the optimization routine is the gradient descent routine, and wherein the output neural network is configured to:
set the latent vector representation of the sample chemical compound as an initial value of the gradient descent routine;
descend along a gradient model of the plurality of latent vector representations to determine a gradient value of a given latent vector representation from among a remaining set of the plurality of latent vector representations;
determine whether the gradient value satisfies a convergence condition; and
designate the given latent vector representation as the candidate latent vector representation in response to the gradient value satisfying the convergence condition.

19. The system of claim 17, wherein the optimization routine is the iterative expansion routine, and wherein the output neural network is configured to:
set the latent vector representation of the sample chemical compound as an initial value of the iterative expansion routine;
select a given latent vector representation from among the plurality of latent vector representations that is proximate to the latent vector representation of the sample chemical compound;
determine a gradient value of the given latent vector representation;
determine whether the gradient value satisfies a convergence condition; and
designate the given latent vector representation as the candidate latent vector representation in response to the gradient value satisfying the convergence condition.

20. The system of claim 17, wherein the optimization routine is the genetic algorithm routine, and the output neural network is configured to:
determine a fitness score for each latent vector representation of at least one set of the plurality of latent vector representations;
select a given latent vector representation from among each of the at least one set based on the fitness score;
perform, for each selected given latent vector representation, a reproduction routine to generate an additional latent vector representation;
determine an additional fitness score associated with the additional latent vector representation; and
designate the additional latent vector representation as the candidate latent vector representation in response to the additional fitness score satisfying a convergence condition.
